(19) United States
(12) Patent Application Publication
(10) Pub. No.: US 2008/0269619 A1
(43) Pub. Date: Oct. 30, 2008

(54) Method and Means for Measuring Systolic Blood Pressure in the Ankle

(76) Inventors: Lars-Goran Lindberg, Linkoping (SE); Bjorn Jonsson, Linkoping (SE)

Correspondence Address: OSTROLENK FABER GERB & SOFFEN, 1180 AVENUE OF THE AMERICAS, NEW YORK, NY, 100368403, US

(21) Appl. No.: 11/719,061

(22) PCT Filed: Nov. 4, 2005
(86) PCT No.: PCT/SE2005/001657

§ 371 Date: Mar. 31, 2008

(30) Foreign Application Priority Data
Nov. 4, 2004 (SE) 0402673-8

(51) Int. Cl. A61B 5/022

(52) U.S. Cl. 600/480; 600/499

(57) ABSTRACT

A flexible pad for measuring systolic ankle blood pressure comprises two or more pairs of light emitting diodes and photo detectors. The pairs are disposed in parallel. The detectors are adapted for detecting light emitted by the respective diode into tissue and reflected from there. The pad further comprises conductor means for providing power to the light emitting diodes from a power source and means selected from conducting means and wireless means for putting the detectors in communication with electronic equipment for detector signal analysis. Also disclosed is the combination of the pad and an inflatable cuff, and a method for measuring systolic ankle blood pressure.

### FIELD OF THE INVENTION

The present invention relates to a method and a means for measuring systolic blood pressure in the ankle.

### BACKGROUND OF THE INVENTION

The determination or monitoring of systolic blood pressure (SBP) is important for the appropriate control of patients with unstable haemodynamics. Invasive measurement by arterial catheters provides correct results but should be avoided, if possible, due to the risk thrombotic events and septicaemia, the formation of haematomae, and cost. Non-invasive methods for measuring or monitoring SBP utilise inflatable (pneumatic) cuffs by which the arterial blood flow in an arm, leg, or finger can be stopped. In the auscultatory or auditory method of Korotkov the pneumatic cuff is wrapped around an upper arm. By inflating the cuff the blood flow in the brachial artery is stopped. A stethoscope is placed over this artery distally off the cuff. On progressive deflation pulsating sounds are detected. SBP corresponds to the manometrically measured pressure in the cuff at which the pulsating sounds can be first heard while the diastolic blood pressure (DBP) corresponds to the lower pressure in the cuff at which the they disappear. Instead of the auscultatory method the Doppler effect of an ultrasound beach back-scattered from moving red blood cells can be used for the measurement and monitoring of SBP. An ultrasound transceiver is placed over the artery distally of the cuff. The onset of arterial blood flow during deflation of the cuff corresponds to SBP, and the Doppler effect generated by the moving red blood cells gives rise to a sound signal in the audible range. Appropriate sites for measuring SBP by the Doppler method are the brachial and radial arteries of the arms and the tibial arteries of the legs. During monitoring the probe must be kept at a correct angle and a sufficient pressure against the skin. A third method for measuring SBP known in the art is the oscillometric method. The oscillating signal emanates from the small, pulse-dependent component in the cuff pneumatic pressure during standard (that is, auscultatory) blood pressure monitoring. The oscillating signal is caused by the mechanical coupling of the blood pressure in the artery which oscillates between SBP and consecutively lower pressure due to ongoing deflation of the cuff. The oscillometric signal is considered to have its maximum amplitude at a mean arterial blood pressure (MAP). From this signal oscillometric devices on the market calculate SBP and DBP using different algorithms most often kept secret by the respective manufacturer. SBP determined by oscillatory techniques is in good agreement with invasively obtained SPB (E O'Brien et al., Brit. Med. J. 322:531-536).

Determination of the systolic blood pressure in the ankle (ASP), and the calculation of the ankle-brachial blood pressure index (ABPI) are important elements in the investigation of leg ischemia. There is increasing interest in these parameters due to the association between a reduced ABPI and generalised arteriosclerotic disease.

Various methods for determining ASP are known in the art. In a standard method, a hand-held continuous-wave pen Doppler device of a frequency of about 8 to 10 MHz and a standard pneumatic cuff for temporarily restricting blood flow are used. Studies of this method (J Stoffers et al., Scand. J. Prim. Health Care 1991; 9:109-114; K W Johnston et al., J. Vasc. Surg. 1987; 6:147-151; F G R Fowkes et al., J. Epidem. Comm. Health 1988; 42:128-133) indicated a variability in ABPI of 10% to 22% in a laboratory setting (95% confidence limitis for single measurement vs. mean of repeated measurements). In clinical practice the variability can be expected to be even higher due to intrinsic problems associated with this technique (S A Ray et al., Br. J. Surg. 1994; 81:188-190; C M Fisher et al., J. Vasc. Surg. 1996; 24:871-875). The examiner has to localise individual ankle arteries with the probe and keep it in the same position and angle during cuff inflation and deflation. Other devices for ASP measurement, such as automatic oscillometric pressure recorders (M Adiseshiah et al., Ann. Royal Coll. Surg. Engl. 1987; 69:271-273; B Y Lee et al., J. Vasc. Surg. 1996; 23:116-122) are potentially less examiner-dependent. While giving comparable results to the Doppler method in healthy individuals, the oscillometric method is considered unreliable in arterial occlusive disease (B Jonsson et al., Clin. Physiol. 2001; 21(2):155-163).

Photopletysmography (PPG) is a further technique by which SBP can be measured. A small area of the skin is illuminated by a light source directly or indirectly, such as via an optical fibre. The radiation, in particular red or infrared radiation, is diffusely scattered in the tissue and blood. A portion is scattered back and can be detected by a photo detector placed adjacent to the illuminated area (reflection mode). With thin tissues, such as on fingertips, it is also possible to detect the scatted light on a skin surface opposite to the illuminated area (transmission mode). PPG is used for non-invasively monitoring pulse rate, respiratory rate, tissue blood perfusion, arterial oxygen saturation, and blood pressure (K Yamakoshi et al., Med. Biol. Eng. Comput. 1982; 20:307-313; R Chawla et al., Acta Anaesth. Scand. 1992; 30:227-230; M Langbaum et al, J. Pediatr. 1994; 125:591-595; U.S. Pat. No. 6,120,459)

### OBJECTS OF THE INVENTION

It is an object of the invention to provide a non-invasive method for measuring systolic blood pressure in the ankle, which is simpler than methods known in the art.

It is another object of the invention to provide a non-invasive method for measuring systolic blood pressure in the ankle, which is more reliable than methods known in the art.

It is a further object of the invention to provide a means for carrying out the method of the invention.

Still further objects of the invention will become apparent from the following short description of the invention and a number of preferred embodiments thereof illustrated in a drawing, and the appended claims.

### SHORT DESCRIPTION OF THE INVENTION

According to the present invention is provided a method for measuring systolic blood pressure in the ankle of a subject, comprising

providing an assembly including an ankle cuff, a flexible measuring pad (“PPG pad”) comprising at least two pairs of light emitting diodes and photo detectors, and an electronic control unit in communication with the pad, the pad being optionally fixed at the cuff;

positioning the pad or, if the pad is fixed at the cuff, the combination of pad and cuff in contact with the skin of the ankle region of the subject so as to dispose one pair in proximity of the anterior tibial artery and substantially parallel with it and the other pair in proximity of the posterior tibial artery and substantially parallel with it and, if the pad and cuff are separate, positioning the cuff around the pad that is in contact with the skin of the ankle region;

inflating the cuff to a pressure sufficient for stopping blood flow through the anterior and posterior tibial arteries;

deflating the cuff while making the diodes emit light;

recording the light reflected from the tissue by the photo detectors during deflation;

recording the cuff pressure during deflation;

analysing the recorded light signal to identify the cuff pressure at which of blood flow in the anterior and/or posterior tibial arteries is resumed.

It is preferred to position the pad or the combination of pad and cuff on the ankle in a manner so as to dispose the photo detectors downstream of the light emitting diodes in respect of the blood flow in said arteries.

It is also preferred to position the pad in respect of the cuff so as to avoid compression of ankle tissue distally of the pad.

According to the present invention is also provided a flexible pad for measuring systolic ankle blood pressure (also termed “PPG pad”), comprising at least two pairs of light emitting diodes and photo detectors disposed in parallel, the detectors being adapted for detecting light emitted by the respective diode into tissue and reflected from there, the pad further comprising conductor means, preferably shielded, for providing power to the light emitting diodes from a power source and means selected from conducting means and wireless means for putting the detectors in communication with electronic equipment for detector signal analysis. It is preferred for the pad to comprise a means for detector signal amplification, such as an AMP board. It is also preferred for the pad to be about rectangular in shape, its short sides extending in a proximal/distal direction when mounted. Preferably the pad is of a size that allows it to be wrapped around at least half of the circumference of the ankle of an adult person, It is also preferred for the SPMP to be divided into two sections of about rectangular shape, the line of division being parallel to the short sides, the sections being termed anterior tibial artery section and posterior tibial artery section. It is though within the ambit of the invention to position the light emitting diodes and/or the photo detectors elsewhere in/on the pad or separate of the pad while providing for light and/or optical signal communication by optical fibers extending from the positions at which the LED's and PD's are intended to be disposed according to the invention to the actual disposition of the LED's and PD's.

According to one preferred aspect of the invention, the light emitting diodes and the photo detectors of the pairs are disposed in a rectangular pattern or in a rectangular network pattern in the pad.

According to a second preferred aspect of the invention the at least two pairs of light emitting diodes and photodetectors are disposed in separate sections of the pad, each section comprising at least one such pair. In a pad with two such pairs, it is preferred for the distance between the light emitting diode and the photodetector in each pair to be smaller than the distance between the light emitting diodes and the photodetectors. In a pad with at least three such pairs, it is preferred for the distance between the light emitting diode and the photodetector in each pair to be smaller than the distance between the light emitting diodes and the photodetectors of the two adjacent pairs that pertain to separate sections.

According to a third preferred aspect of the invention, the pad or the combination of pair and cuff is provided with a mark indicating its correct position in regard of the anterior margo tibiae.

According to a fourth preferred aspect of the invention each light emitting diode and photodetector pair comprises an additional light emitting diode, preferably disposed in a linear manner on the cuff and with the photodetector positioned in-between the light emitting diodes.

According to a fifth preferred aspect, at least three pairs, each preferably comprising an additional photodetector disposed in the manner of the fourth preferred aspect, are arranged in each section of the pad.

According to a sixth preferred aspect a combination of the pad of the invention and an inflatable ankle cuff is provided. Preferably, the pad is fixed to the cuff, either permanently or, more preferred, releasingly, such as by Velcro® means.

According to the present invention is also provided a system for measuring systolic blood pressure in the ankle of a subject, comprising the measuring pad of the invention, an ankle cuff that may be fixed to the cuff or be separate, pump means for inflating and deflating the cuff, gauge means for recording the pressure in the cuff, electronic means for amplification of photodetector signals, computing means for analysing the amplified signal, and power supply means.

The invention will now be explained in greater detail by reference to a drawing illustrating several preferred but not limiting embodiments thereof.

### DESCRIPTION OF THE FIGURES

FIG. 1 is a rough sketch of a transverse section through a leg at its plane of smallest circumference at which the PPG pad is mounted;

FIG. 2 is a preferred embodiment of the PPG pad of the invention, in a top view of its application face;

FIG. 3 is a transverse section along line A-A through the PPG pad of FIG. 2 in combination with a inflatable cuff;

FIG. 4 is the PPG pad of FIG. 2, disposed on the cuff, in the same view as in FIG. 2;

FIG. 5 is a schematic view of a preferred embodiment of the system of the invention for measuring ankle systolic blood pressure;

FIG. 6 is a scheme illustrating the circuitry of the embodiment of FIG. 5;

FIG. 7 is a diagram showing raw PPG pad sensor signal amplitude v. cuff pressure traces for the anterior tibial artery and the posterior tibial artery during deflation obtained with the system of FIG. 5;

FIG. 8 is a diagram showing the amplitude v. cuff pressure traces refined by computer analysis of the corresponding traces of FIG. 7, the identification of the first true pulsatile signal in the anterior tibial artery (ATA 1) and the posterior tibial artery (PTA 1), and the corresponding cuff pressure;

FIG. 9 is a graph comparing systolic ankle pressure data obtained by the method of the invention and from state-of-the-art CW Doppler measurements;

FIG. 10 is a graph illustrating the effect of incorrect positioning of the PPG pad of the invention on PPG measurements of systolic blood pressure in the ankle according to the invention; and

FIG. 11 is a graph comparing systolic blood pressure in the ankle obtained by the method of the invention and by an invasive method.

### DESCRIPTION OF PREFERRED EMBODIMENTS

### Example 1

### Anatomic Considerations

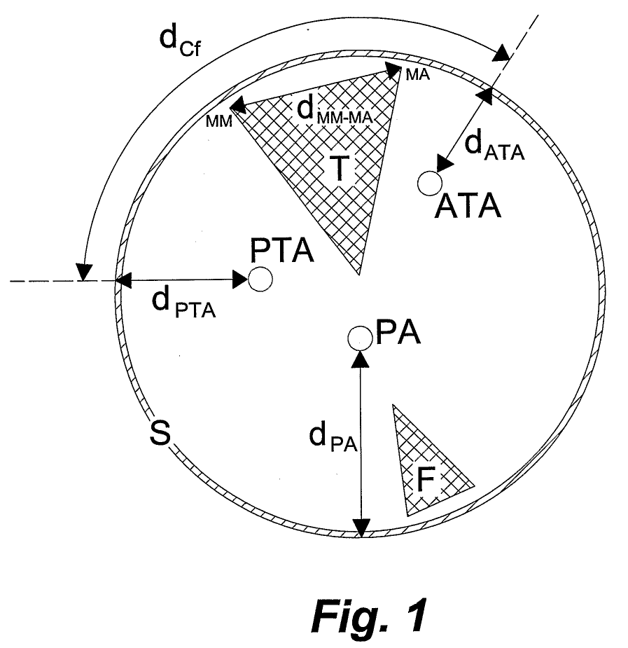

FIG. 1 illustrates schematically a transverse section through a right leg at its plane of smallest circumference at which the PPG pad is mounted, with tibia T, fibula F, anterior tibial artery A, posterior tibial artery PTA, peroneal artery PA, and skin S shown.

Leg circumference at its most narrow ankle section was measured in 84 individuals (Table 1; age 18-85 years, median 67 years; 52 men, 32 women; 66 patients, 18 healthy volunteers; both legs).

TABLE 1
Leg circumference and distance of arteries and their cutaneous
projections in an ankle cross sectional plane

| | Range | Mean | SD |
|---|---|---|---|
| Leg circumference (161 legs in | 170-265 | 218 | 20 |
| 84 subjects*⁾, mm | | | |
| Distance artery/cutaneous proj. | | | |
| (both legs in 20 subjects), mm | | | |
| Anterior tibial a. | 9-26 | 15 | 4 |
| Posterior tibial a. | 8-24 | 15 | 4 |
| Peroneal a. | 13-36 | 22 | 5 |

*⁾Legs with ankle edema (n = 7) excluded

In 20 of the legs the distance between the skin surface and the major ankle arteries, d_{ATA} for the anterior tibial artery, d_{PTA} for the posterior tibial artery, d_{PA} for the peroneal artery, was measured by colour-coded Duplex ultrasound (Table 1; ATL HDI5000 with an L 7-4 linear probe, Philips, Netherlands). In addition, the distance d_{Cf} between the cutaneous (skin designated S in FIG. 1) projections of the anterior ATA and posterior PTA tibial arteries in the cross sectional plane was measured in the right leg of 34 other subjects of the study group (19 patients, 15 healthy volunteers) by 8 MHz or 10 MHz hand-held Doppler ultrasound probes (Table 1). The width of the tibial bone in the same plane, measured as the distance d_{MM-MA}, between the margo anterior MA, the easily palpable frontal edge of the tibia, and the margo medialis MM, the lateral (inward) edge of the tibia, as calculated from nuclear magnetic resonance tomography (MR) images of one leg in seven healthy volunteers (26-52 years; five men, two women). A mean of 25 mm (SD 2, range 23-28 mm was obtained for d_{MM-MA}.

### Example 2

### PPG Pad Design

Based on the anatomical findings, the features of a probe for measuring systolic blood pressure at the ankle were set to allow a variation of more than two standard deviations from the mean values in ankle circumference and in the distance between the arteries. The thickness of the tibial bone and its relation to the arteries, based on the MR scans, were also taken into account in setting the distance between the two channels. By the design of the probe, the considerably deeper located peroneal artery was assumed to be essentially beyond the detection range.

The positioning of the probe pays regard to the asymmetry of the arteries relative to the easily palpable frontal edge of the tibia. A marker on the probe (or one outside the cuff if a fixed probe/cuff combination is used) is positioned in line with this anatomical landmark. Since the anatomy of the right ankle is a reflection of the left and the probe prototype is asymmetrical, it has to be rotated 180° when right and left legs are examined, respectively.

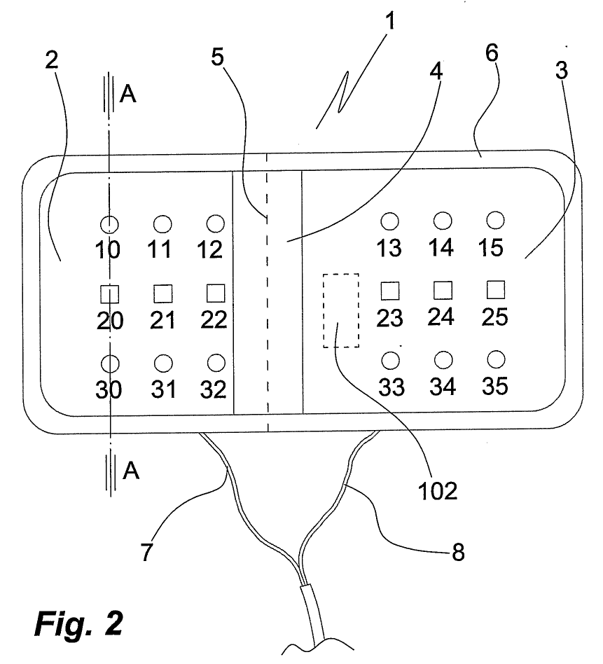

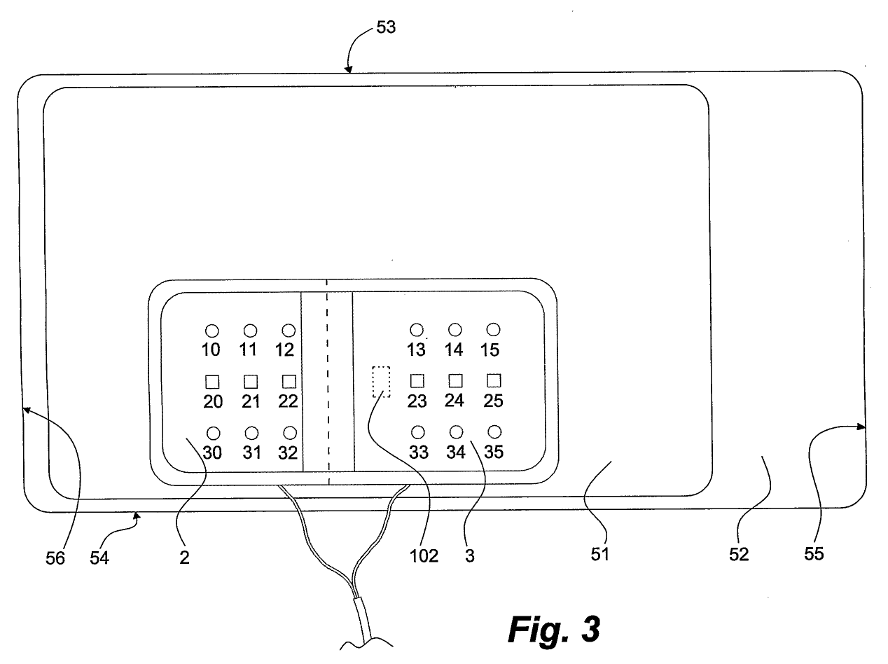

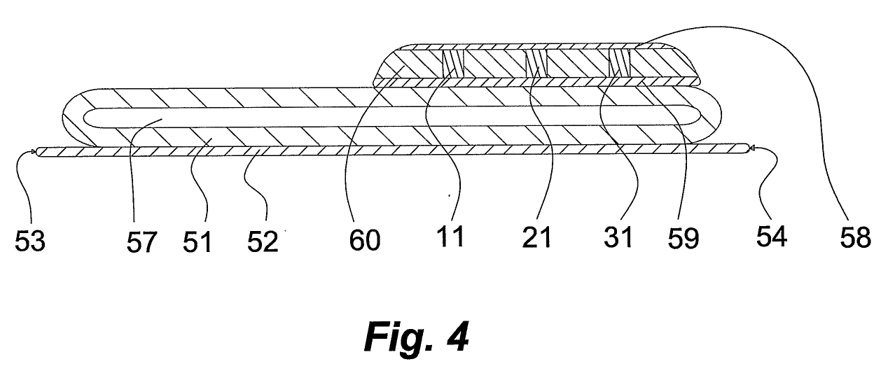

The about rectangular flexible PPG pad 1 according to a preferred embodiment of the invention illustrated in FIGS. 2 and 3 is shown in a disposition for application to the left ankle of a person. The hatched line 5 indicates how the pad 1 is aligned with the margo anterior tibiae; a corresponding mark is provided at the rear (outer, when applied to the ankle) face of the pad 1 or the cuff, if a fixed combination of pad 1 and a cuff 52 (FIG. 4) is used. When the pad 1 is applied to the left ankle, the hatched line 5 will abut the skin at the front of the left leg in close proximity of the tibia, with which it is aligned. The pad 1 comprises a sheet of soft, flexible silicone material 2, 3, 4, 6 in which a number of first 10, 11, 12, 13, 14, 15 and second 30, 31, 32, 33, 34, 35 light emitting diodes (LED) and photo detectors (PD) 20, 21, 22, 23, 24, 25 are embedded in three rows perpendicular to the hatched line 5, two LED rows and one PD row. Additionally embedded is an AMP board 102 for photodetector signal amplification. When applied to the subject the portion of the pad 1 on the top of FIG. 2 becomes the proximal portion; correspondingly the portion at the bottom becomes the distal portion. Half of the LEDs in the first or proximal LED row 10, 11, 12, 13, 14, 15 and the LEDs in the second or distal LED row 30, 31, 32, 33, 34, 35 are disposed on either side of line 5. Thus LEDs 10, 11, 12 of the first, proximal LED row and LEDs 30, 31, 32 of the second, distal LED row become disposed in proximity of the anterior tibial artery ATA; they are termed ATA LEDs. The other half of the LEDs (first or proximal LED row: 13, 14, 15; second or distal LED row: 33, 34, 35) become disposed in proximity of the posterior tibial artery PTA) they are termed PTA LEDs. Similarly, half of the photo detectors 20, 21, 22, 23, 24, 25 are arranged to the left of line 5, in which disposition they will primarily detect light reflected from the anterior tibial artery area; consequently they are termed ATA PDs 20, 21, 22. The other half of photo detectors 20, 21, 22, 23, 24, 25 are arranged to the right of line 5, in which disposition they will primarily detect light reflected from the posterior tibial artery area; consequently they are termed PTA PDs 23, 24, 25. The left and right (in relation to line 5) PPDs and photo detectors define left (anterior) and right (posterior) pad sections 2 and 3, respectively, which are separated by an intermediate section 4 that may be of a thinner flexible material such as, for instance, a textile material. Sections 2, 3, 4 are surrounded by a narrow circumferential border 6. The LEDs and PDs in the left and right portions of a row are disposed in an equidistant manner, the distance between them being about 15 mm. The distance of the innermost members (those closest to line 5) in a row is about 50 mm but the distance of the innermost member of a left row from line 5 is substantially shorter (about 15 mm) that the corresponding distance of the innermost member of a left row (about 35 mm); this reflects about the disposition of the corresponding arteries in relation to the front edge (or line 5) of the tibia. The distance between rows in a proximal/distal direction is about 20 mm.

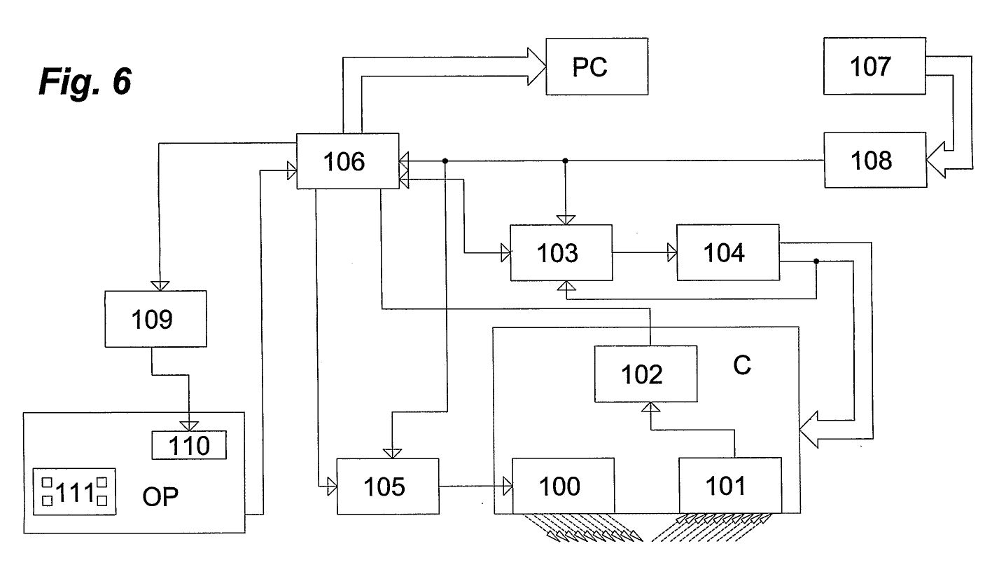

The flexible PPG pad of FIGS. 2 and 3 is manufactured in the following way. A cast form for the pad body is made from wood. The cast material, a two-component silicon rubber, Rhodorsil RTV 1556 A and B (Sikema, Sweden; hardness upon curing: 28±3 Shore A) and black pigment, Intensive Black 886, (Uterm, Sweden) is mixed at room temperature in a plastic container, and the mixture is poured into the form. Incorporating black or other pigment light reduces interference by light from outside. Air bubbles are removed by placing the form in a steel container, which is evacuated. The polymer is hardened in an oven. The pad body 60 thus produced comprises windows that mirror the LED/PD pattern of the pad 1. The LED's and the PD's are accordingly put into the respective windows and fixed by gluing with transparent silicone glue, Glass Silicone Transparent (Dana, Denmark). In an additional window the probe AMP board 102 is placed and fixed by gluing. The photo diodes are connected to the probe AMP board 102 by electrical leads disposed on the rear side of the body 60. The output lead 8 from the AMP board 102 and the LED power supply lead 7 are inserted into a shielded cable 78, STC-36T-12 (Vishay Measurement Group, Germany). A cable connector, HR10A (ELFA, Sweden) is connected to free end of the shielded cable for connection to a PPG board 106 (FIG. 6). After control of optical and electrical functions, a sheet of silicone rubber 59 is glued to the rear side of the body 60 to protect the components (PDs, LEDs, probe AMP board, electrical conductors). A thin sheet of transparent silicone rubber 58 is glued to the front side of the pad so as to cover it completely. The flexible PPG pad 1 thus manufactured is about 15 mm thick. In FIG. 3 its thickness is greatly exaggerated for reasons of clarity. The same is true for the inflatable cuff 51, the lumen of which is indicated by reference number 57. If desired, the flexible PPD pad 1 may be glued or otherwise fixed to the cuff 51 at the inside thereof. Suitable LEDs are, for instance, Siemens (Germany) SFH 420 (880 nm). Suitable photodetectors are, for instance, Siemens (Germany) BPW34. A suitable cuff for blood pressure measurements can be obtained from OMRON Healthcare UK (Henfield, West Sussex, UK).

FIG. 4 illustrates the disposition of the flexible PPG pad of FIGS. 2 and 3 in relation to an inflatable cuff 50 of about rectangular shape having a proximal (top) edge 53, a distal (bottom) edge 54, and left 56 and right 55 edges. The cuff 50 may be a state-of-the-art cuff used for blood pressure measurements with an inflatable portion 51 and a non-inflatable flap portion 52 provided with a fixation means (not shown) such as Velcro® tape. The PPG pad 1 of the invention is disposed close to the distal edge 54 of the cuff. When inflated, the cuff 50 thus restricts arterial blood flow proximally (upstream) of the flexible PPG pad 1 but may additionally restrict blood flow at the application area of the pad.

### Example 3

### System for Measuring Systolic Ankle Blood Pressure

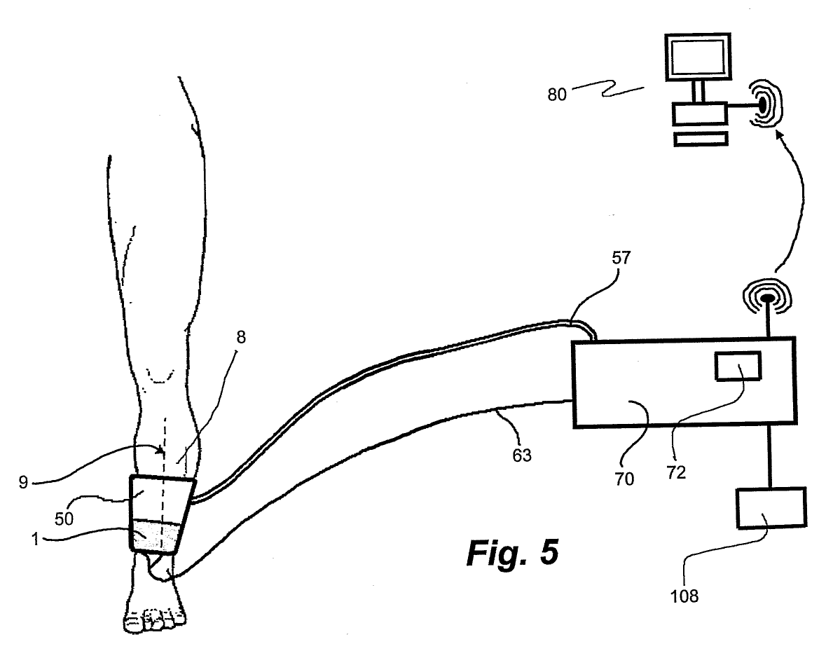

An embodiment of the system for determining systolic ankle blood pressure of the invention is schematically illustrated in FIG. 5. The system comprises a flexible PPG pad 1 of the invention and an inflatable cuff 50; both are shown mounted to the ankle of a right leg 8 in which the front edge 9 of the tibia is indicated. The flexible PPG pad 1 is positioned between the cuff 50 and the ankle and thus covered by the cuff 50. A shielded cable 63, which extends from the PPG pad 1, and a tube 57 for pressurised air, which extends from the cuff 50, are connected with a PPG/cuff pressure control unit 70, which comprises a panel 72 with a screen and operator input means. Power is supplied to the control unit 70 by a power source 108. The control unit 70 is in wireless communication with a personal computer 80 in which photodetector signal amplitude and cuff pressure data are analysed and stored.

### Circuitry for Assembly for Measuring Ankle Systolic Pressure.

The circuitry of the system of FIG. 5 for measuring systolic ankle blood pressure measurement is shown in greater detail in FIG. 6. It comprises at least one light source 100 and at least one photo detector 101 in a flexible PPG pad which is combined with a inflatable cuff C. The photo detector 101 is electrically connected to an AMP board 102 in the pad for amplification of the weak detector signal from which it is sent to a PPG board 106 that hierarchically controls the system. The PPG board comprises an analog to digital converter. The converted LED signal is sent to a PC in which it is analysed and stored. Via a pump board 103 the PPG board 106 controls a pump/valve assembly for inflating and deflating the cuff. The pump board 103 comprises pressure sensors the signals of which are fed to the PPG board. A safety circuit opens a magnetic valve at a selected pressure, which can be adjusted by a potentiometer. Typically the safety limit will be set at about 400 mm Hg. The PPG board 106 also controls the light source 101 via an LED driver board 105. A bedside operator panel OP comprising a display 110 and a control panel 111 connected to the PPG board 106 allows the operator to carry out the measurement. The control panel 111 comprises input means for start/reset of measurement, start/reset of cuff pressure, LED gain, etc. The LED signal and other data is presented on a display 110. The signal from the PPG board 103 fed to the display is converted in a display board 109 that also comprises latch and driver means for the display. Power from an external power supply 107 is filtered and distributed via a power board 108.

### Example 4

### PD Signal Analysis Algorithm. Determination of True First Systolic Peak

The signal analysis used to extract the local PPG peak that corresponds to the pressure in the occlusion cuff just below the systolic pressure (when arterial blood starts to pass the cuff) is performed in three steps:

a) The information reduction step is constituted by a differentiating filter (a simple first order differentiating convolution kernel), followed by a 10^{th} order band pass Butterworth filter with cut frequencies at 4 Hz and 9 Hz applied to the signal in order to retain only the pulsatile features of the signal.

b) Peak extraction step. Extracts PPG signals corresponding to true heart beats. PPG peaks that correspond to true beats are determined by analysis of the amplitude of the signal appearing within intervals determined by, in part, the assumption that a reasonable heart rate at rest does not exceed 100 beats per min and, in part, by the most frequently occurring amplitude peak distance, i.e., the heart rate. The amplitude of the signal is determined by using a 16^{th} order Hilpert transformer filter.

The Hilbert transformer is a filter that approximates the discrete Hilbert transform

$${f}_{H i} ( x ) = \frac{1}{π} {\int }_{- ∞}^{∞} \frac{f ( τ )}{τ - x} d τ ,$$

of a signal

f(x)

which is an integral part of the analytic signal

The analytical signal is in turn used to derive the amplitude of the signal

f(x)

as the instantaneous amplitude of

f(x)is|f_{A}(x)|(={square root over ([f(x)]²+[f_{Hi}(x)]²))}.

c) Peak selection step. Selects the peak that corresponds to the systolic pressure. The PPG peak is selected that exhibits the greatest difference in amplitude compared to the peak next in line, and which is followed by a predetermined number of peaks, typically 15-20, depending on sampling time.

It is, of course, also possible to visually determine the ankle systolic pressure by identifying in the PD v. cuff pressure trace the first peak in a row of peaks of increasing amplitude and about equal spacing.

Another method for identifying the first true peak in a PD/cuff pressure diagram is by gating. The peak with the largest or second largest amplitude (reference peak) is identified. Peaks smaller than a set fraction of the reference peak are disregarded from. The first peak having an amplitude equal or higher than said fraction is considered to be the first true peak. Gating can be used as such or in combination with other methods.

### Example 5

### Ankle Systolic Blood Pressure Measurement Procedure

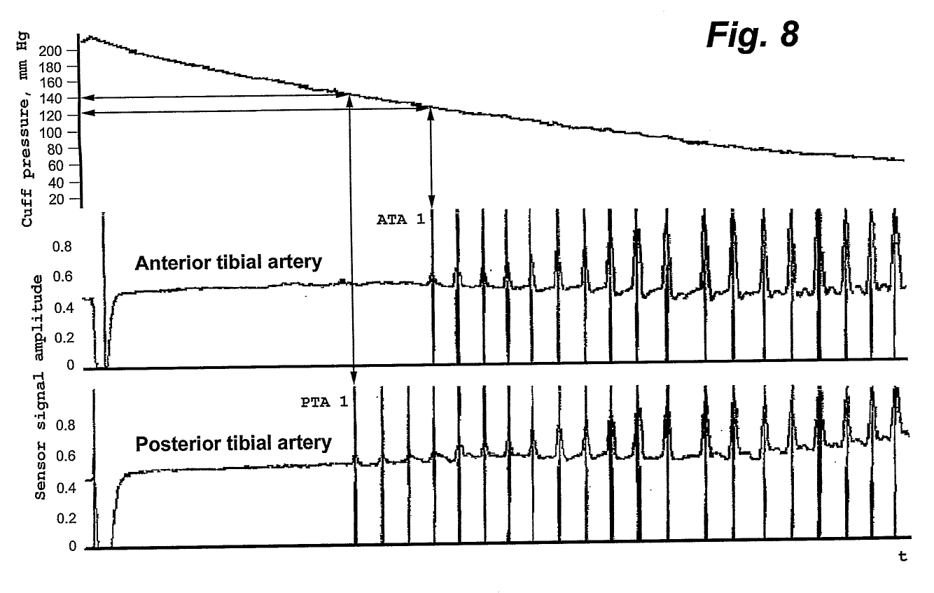

The PPG pad 1 of the invention is positioned at the ankle with the marker corresponding to line 5 in line with the palpable front edge MA of the tibia. The cuff 50 is then applied on the pad in a manner that the lower edge of the cuff 50 superimposes the lower edge of the pad 1. The person on which the measurement is performed is placed in a supine position on a bed. The measurement is started by inflating the cuff 50 while monitoring the PD signals. The cuff is inflated above the pressure range at which the PD signals become essentially flat, that is, at which blood ceases to flow in the anterior ATA and posterior PTA tibial arteries. Monitoring is continued during the entire procedure of inflation and deflation of the cuff 50, which lasts about 120 seconds (cuff deflation rate approx. 3 mm Hg/s). On release of the pressure in the cuff the signals from the anterior and posterior tibial arteries, respectively, appear consecutively (FIG. 8) or about at the same time, depending on the condition of the particular patient.

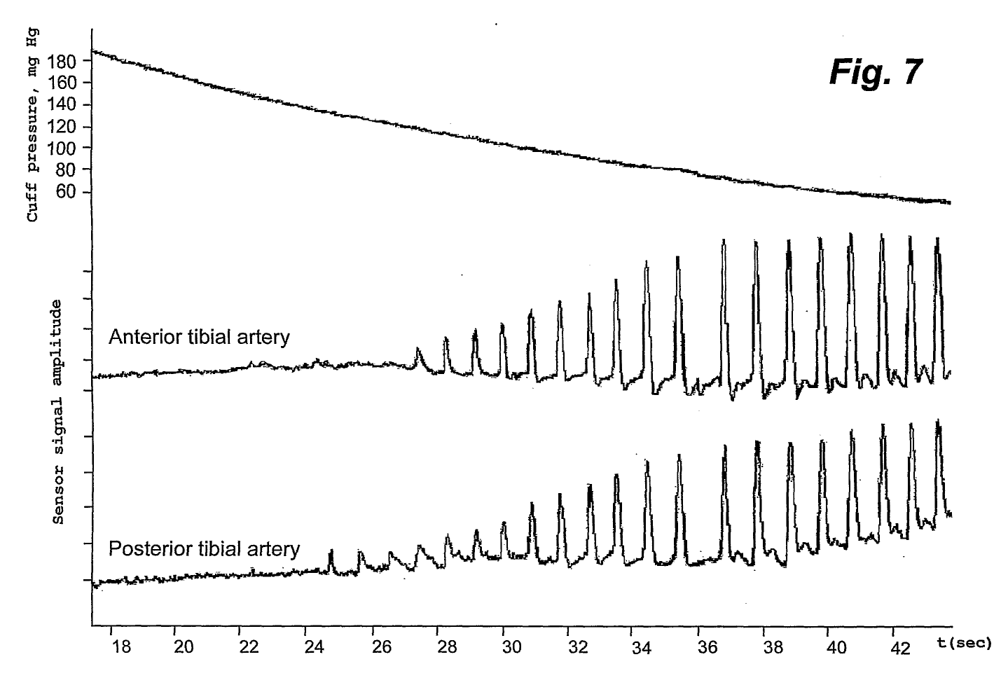

The raw PD amplitude signals from the anterior and posterior tibial arteries, respectively, are shown in FIG. 7 in relation to cuff pressure during deflation. The corresponding PC-analysed signals are shown in FIG. 8, in which also the peak corresponding to the first true pulse (heart beat) is identified in each trace. The cuff pressure for this peak corresponds to the ankle systolic pressure for the anterior and posterior tibial artery, respectively.

### Example 6

### PPD Pad Systolic Ankle Blood Pressure Measurement Evaluation—Non-Invasive Measurements

For comparison with the standard Doppler technique, 20 healthy volunteers (24-55 of age, 15 men) were examined. All subjects were placed supine on a bed at room temperature. The measurements were performed at the right ankle after five minutes of rest. Along with the PPG recordings, CW Doppler pressure measurements in the arteria dorsalis pedis and posterior tibial arteries were performed simultaneously using an 8-MHz handheld Doppler (MD-8, Sonotech GMBH, Schwaben, Germany). The systolic pressure was read on the aneroid sphygmomanometer at the closest 2 mm Hg. Four consecutive measurements were performed, two in each ankle artery. The PPG signals were stored on the PC hard disc for later analysis. During the procedure, the PPG-derived pressures were not available to the examiner. The influence of incorrect probe positioning was tested in 18 of the subjects. Measurements were made with the cuff and pad rotated 2 cm medially and laterally from the correct position, respectively. Two measurements were performed in each position, one in each ankle artery, using the same procedure as above. A PPD pad with the LED/photo detector configuration of FIGS. 2 and 3 was used.

The simultaneous CW Doppler measurements were carried out with an aneroid sphygmomanometer (Maxi-Stabil 3, Welch Allyn, N.Y., USA) connected to the pressure regulator unit.

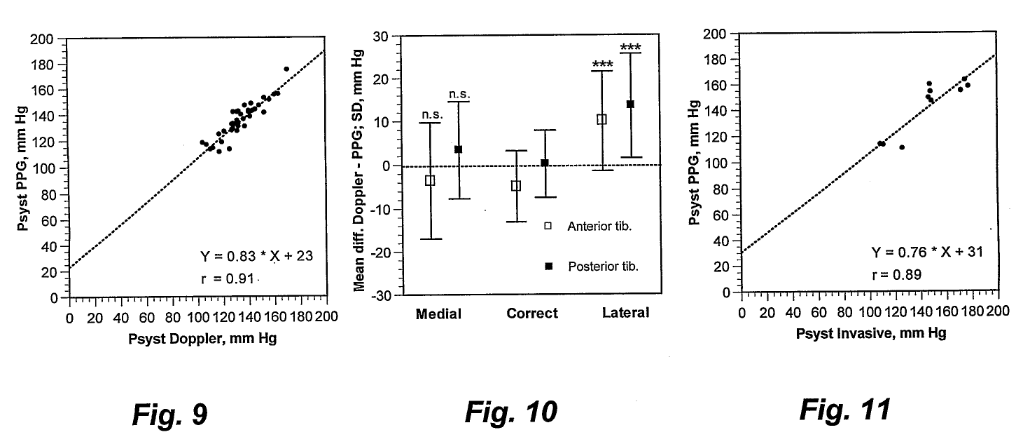

Visual analysis of the reappearance of PPG pulsations during cuff exsufflation was possible in all 80 measurements, despite numerous movement artefacts and/or noise. The results are shown in Table II and in FIG. 9 (mean of two measurements in each position). Rotating the combination of cuff and pad two cm laterally from the central, predetermined “correct” position produced a mean difference between CW Doppler and PPG results in the anterior tibial artery of 10.1 mm Hg (SD 11.5). This compared to −4.9 mm Hg (SD 8.4) with the “correct” position (n=17 (one measurement excluded due to technical errors), p<0.001). In the posterior tibial artery, the mean differences were 13.6 mm Hg (SD 13.1) and 0.2 mm Hg (SD 7.6) respectively (n=18, p<0.001). A 2 cm medial rotation did not alter the mean error significantly, but the standard deviations increased (mean differences CW Doppler—PPG −3.6 (SD 13.3) and 3.4 (SD 11.1) mm Hg for the anterior and posterior arteries, respectively) (FIG. 10).

### Example 7

### PPG Pad Ankle Systolic Pressure Measurement Evaluation—Invasive Measurements

Ten neurosurgical or thoracic patients at the intensive care unit (40-75 years old, three males) with an arterial line put in for clinical monitoring purposes were included. None had a diagnosis of peripheral arterial disease; all had palpable foot pulses. A 1.1×45 mm cannula (BD, UK) was inserted in the arteria dorsalis pedis on the dorsum pedis. The cannula was fixed and connected to tubings that included devices for calibration against heart level, and a damping device. The line was connected to a pressure-monitoring device (HP M 1006A/B pressure module and HP Viridia monitor, Hewlett Packard, USA). The pressure curves were carefully checked for artefacts (flushing technique), and a damping device was used when necessary to avoid under-damping (“ringing”). For practical reasons, PPG recordings were performed in the contralateral leg. Doppler systolic pressures in the arteria dorsalis pedis arteries of right and left leg were examined. Only subjects with a pressure difference not exceeding 5 mm Hg between legs were accepted. The intra-arterial systolic pressure from the arterial line was recorded during exsufflaton of the cuff. A minimum of six measurements were performed in each patient. In comparison with systolic ankle pressure measured intra-arterially in the arteria dorsalis pedis, PPG (by visual examination) underestimated systolic pressure in the contralateral anterior tibial artery by 4.5 mm Hg (SD 11.2), (FIG. 11).

Statistics. Continuous data are presented as their means and standard deviations (SD). 95% confidence intervals were calculated for mean differences. Pearson correlation coefficient (r) were calculated where appropriate. Comparisons were performed using Student's t-test for paired samples. P-values of less than 5% were considered statistically significant. All calculations were performed using SPSS for Windows version 11.0 (SPSS Inc., USA).

### Example 8

### Simultaneous Measurement of Ankle and Arm Systolic Blood Pressure

Ankle systolic pressure measurements with the pad of the invention were carried out in combination with the simultaneous measurement of systolic blood pressure in the arm to obtain the so-called Ankle Brachial Index (ABI). An ABI of >0.95 indicates a healthy state. Ankle pad measurements of the invention substituted the standard Doppler ultrasound technique. A pad of a design similar to that of the ankle pad of the invention but simplified, there being no need to discern between pressures in various brachial arteries, can be used for brachial systolic blood pressure measurement. The measurement of ABI can be accomplished with both pads connected to a single control unit corresponding to control unit 70 of FIG. 5. The computer 80 compares the measured ankle and brachial pressures for calculation of ABI.

1. A method for measuring systolic blood pressure in the ankle of a subject, comprising
providing an assembly including an ankle cuff, a flexible measuring pad comprising two pairs or more of light emitting diodes and photo detectors, and an electronic control unit in communication with the pad, the pad being optionally fixed at the cuff;
positioning the pad or, if the pad is fixed at the cuff, the combination of pad and cuff in contact with the skin of the ankle region of the subject so as to dispose one pair in proximity of the anterior tibial artery and substantially parallel with it and the other pair in proximity of the posterior tibial artery and substantially parallel with it and, if the pad and cuff are separate, positioning the cuff around the pad that is in contact with the skin of the ankle region;
inflating the cuff to a pressure sufficient for stopping blood flow through the anterior and posterior tibial arteries;
deflating the cuff while making the diodes emit light;
recording the light reflected from the tissue by the photo detectors during deflation;
recording the cuff pressure during deflation;
analysing the recorded light signal to identify the cuff pressure at which of blood flow in the anterior and/or posterior tibial arteries is resumed.

2. The method of claim 1, comprising positioning of the pad or the combination of pad and cuff on the ankle in a manner so as to dispose the photo detectors downstream of the light emitting diodes in respect of the blood flow in said arteries.

3. The method of claim 2, comprising positioning the pad in respect of the cuff so as to avoid compression of ankle tissue distally of the pad.

4. A flexible pad for measuring systolic ankle blood pressure, comprising two or more pairs of light emitting diodes and photo detectors, said pairs being disposed in parallel, the detectors being adapted for detecting light emitted by the respective diode into tissue and reflected from there, the pad further comprising conductor means for providing power to the light emitting diodes from a power source and for putting the detectors in communication with electronic equipment for detector signal analysis.

5. The pad of claim 4 comprising a means for detector signal amplification.

6. The pad of claim 5, wherein the detector signal amplification means comprises an AMP board.

7. The pad of claim 4 of about rectangular shape, its short sides being designed to extend in a proximal/distal direction when mounted on the leg of a patient.

8. The pad of claim 4, of a length allowing it to extend around at least half of the circumference of the ankle of an adult person.

9. The pad of claim 7 comprising two sections of about rectangular shape, the line of division being parallel to the short sides and intended for disposition along the margo anterior tibiae in a mounted position.

10. The pad of claim 9, wherein each section comprises one or more pairs of light emitting diodes and one or more pairs of photodetectors.

11. The pad of claim 10, wherein the disposition of the light emitting diodes and the photodetectors allows separate detection of light reflected from the anterior tibial artery and from the posterior tibial artery.

12. The pad of claim 11, wherein the distance between the light emitting diode and the photodetector in a pair is smaller than the distance between light emitting diodes and photodetectors in different pairs.

13. The pad of claim 11, wherein said disposition includes that of the free ends of optical fibers extending from said light emitting diodes and/or photodetectors.

14. The pad of claim 4, wherein the light emitting diodes and the photo detectors and/or the corresponding free ends of the optical fibers are disposed in rectangular pattern or a rectangular network pattern.

15. The pad of claim 4, comprising six pairs of light emitting diodes and photodetectors.

16. The combination of the pad of claim 4 and an inflatable ankle cuff.

17. The combination of claim 16, wherein the pad is fixed to the cuff.

18. The combination of claim 17, wherein in the pad is releasingly fixed to the cuff.

19. A system for measuring systolic blood pressure in the ankle of a subject, comprising the measuring pad of claim 4, an ankle cuff fixed to the cuff or separate of it, pump means for inflating and deflating the cuff, gauge means for recording the pressure in the cuff, electronic means for amplification of photodetector signals, computing means for analysing the amplified signal, and power supply means.

20. A system for determining the Ankle Brachial Index of a subject, comprising the system of claim 19, means for determining brachial systolic blood pressure, and means for calculating ABI.

21. The system of claim 20, wherein the means for determining brachial systolic pressure include a brachial measuring pad comprising at least one pair of light emitting diode and photodetector, pump means for inflating and deflating the brachial cuff, gauge means for recording the pressure in the brachial cuff, electronic means for amplification of photodetector signals from the brachial measuring pad, computing means for analysing the amplified signal from the brachial cuff.

22. The pad of claim 12, wherein said disposition includes that of the free ends of optical fibers extending from said light emitting diodes and/or photodetectors.
